(19) United States
(12) Patent Application Publication
(10) Pub. No.: US 2023/0381475 A1
(43) Pub. Date: Nov. 30, 2023

(54) APPARATUS AND METHOD FOR SURGICAL PREPARATION

(71) Applicant: Prep Tech, L.L.C., Westlake, LA (US)

(72) Inventors: Mark R. Moore, Westlake, LA (US); Noam Gavriely

(73) Assignee: Prep Tech, L.L.C., Westlake, LA (US)

(21) Appl. No.: 17/804,798

(22) Filed: May 31, 2022

(51) Int. Cl. A61M 35/00

(52) U.S. Cl. CPC A61M 35/10; A61M 2205/0205; A61M 2210/086

(57) ABSTRACT

A device used for applying an antiseptic preparation to a patient outside of a sterile environment. In one embodiment, a loose-fitting bag is provided which encloses a patient appendage. In another embodiment, a loose-fitting bag is provided which encloses all or part of the lower half of a patient. A textured surface is provided on the interior surface of the bag. The bag includes one or more removable drainage pouches attached to resealable lower ports. The bag further includes side closures which can be ruptured to allow access to the disinfected appendage or appendages. After disinfection, the antiseptic preparation is drained and sealed in the drainage pouches. The drainage pouches are then removed and discarded.

### FIELD OF THE INVENTION

This present invention relates to an apparatus and method for providing a sterile environment for applying antiseptic prior to surgery, and cleaning wounds prior to, during, and after surgery.

### BACKGROUND OF THE INVENTION

Antiseptics are used prior to conducting the surgery to clean and disinfect the surgical site in order to reduce surgical site infections. Types of antiseptics include alcohols, iodine or iodine-containing compounds and chlorhexidine gluconate among others. There are two primary types of iodine-containing compounds, tincture of iodine, and iodophors. Tincture of iodine is an alcohol solution and was one of the first antiseptics used. However, iodophors or chlorhexidine and alcohol solutions are more commonly used today to prepare a surgical site for surgery.

The most common aseptic technique for disinfecting a surgical site requires application of an antiseptic solution immediately prior to the surgical procedure in the sterile operating theater after the patient has been anesthetized. When performing surgery to the extremities, i.e., leg or arm, the entire extremity is typically cleaned with the antiseptic solution. A sponge is immersed in the antiseptic solution, then applied to an area of the extremity with a scrubbing action and then discarded. A new sponge is immersed into the antiseptic solution and applied to a different area of the extremity. This process is then repeated until the entire area has been scrubbed. The scrubbing action physically dislodges bacterial colonies. Once the scrubbing is completed, another antiseptic solution is reapplied with a sponge in a painting action using a new sponge with each repeated application. After this procedure the surgical personnel must wait at least five to seven minutes before beginning the surgical procedure. The delay is required to allow the antiseptic solution to disinfect the surgical site.

There are several problems with the prior art procedure for disinfecting a surgical site. During the application of the antiseptic solution to the extremity, numerous sponges are required to be used and discarded, thus creating medical waste that must be handled and properly discarded. There is also a possibility that the surgical site will not be completely covered, thus creating a potential for bacteria and other contaminants remaining during surgery and serving as a potential source for post-surgical infection. The application of the antiseptic solution is prone to splashing and uncontrolled spills. Excess antiseptic solution spills create a potential safety hazard. Even where the application of the antiseptic solution is controlled, the drainage of excess solution can create a safety hazard. Furthermore, disposal of excess antiseptic solution can create compliance concerns because it must comply with biohazard disposal regulations.

The prior art procedure for applying the antiseptic solution is also costly and time consuming. Operating theaters are expensive to maintain and operate. The time that the antiseptic solution takes to be effective costs the patient and the hospital a significant amount of money. The delay also slows the throughput of the operating theater, thereby raising the cost of the procedure to the hospital and the operating staff.

The prior art procedure puts the patient at an increased risk of morbidity and mortality by increasing the amount of time the patient is under anesthesia.

Various prior art devices and methods have attempted to remedy these problems. But none of the prior art has adequately streamlined the application, drainage, and disposal of a surgical antiseptic prior to surgery.

For example, U.S. Pat. No. 2,661,739 to Caskey discloses a casing which is made from elastic material to fit against an extremity to hold an absorbent material against a wound. The casing is surrounded by a fabric jacket to hold the casing and absorbent material in place and exclude contamination from the environment. However, the casing is only disclosed to be used after a wound has occurred and it does not disclose using the device to pre-treat an area prior to surgery. Additionally, it does not allow for the scrubbing of medicine against the skin through the device.

U.S. Pat. No. 6,992,233 to Drake, et al. discloses a delivery system for a flowable medicine to a wound in a confined area. Flowable medicine is contained in a strip which is adhesively applied to the skin such that the medicine is delivered to the wound. The flowable medicine is released when removable seal is removed. Drake does not disclose using the device for application of medicine prior to a wound occurring or to a large area of the body. It is limited to the area covered by the strip. Drake also does not disclose scrubbing the medicine against the skin through the device.

U.S. Pat. No. 6,664,434 to Cominsky discloses using a sealed bag around a wound to contain bodily fluids. The device includes an absorbent layer to absorb the bodily fluids exuding from a wound. It does not disclose using the bag prior to the wound or introduction of an antiseptic fluid within the bag. It also does not disclose the scrubbing of an antiseptic through the device.

As yet another example, U.S. Pat. Nos. 8,758,310; 8,403,898; 9,409,005; 10,188,842; and 10,470,842 and U.S. Publication No. 2019/0184146 to Moore disclose devices and methods for applying an antiseptic solution to a surgical site prior to surgery, but outside the operating theater. However, they do not disclose a means for draining of excess fluid in a removable and sealable pouch for disposal.

The prior art fails to disclose or suggest an application with an adequate means for preparing a patent's skin prior to surgery and efficiently draining and disposing excess solution. Therefore, it is desirable to have a device and method for facilitating application of antiseptic solution in a non-sterile environment with a removable and resealable drainage means.

### SUMMARY OF THE INVENTION

An apparatus and method for sterilizing a portion of a patient prior to surgery is described.

In one embodiment, the apparatus is an application bag which is sealed on three sides and open on a fourth side. In another embodiment, the apparatus is an application bag which includes an open end and two closed leg portions. In yet another embodiment, the apparatus is an application bag which includes two open ends and one closed leg portion.

The apparatus includes a textured interior surface for dislodging bacterial colonies. The apparatus also includes one or more removable drainage pouches. In use, after the bag is sealed to the patient, the application bag is manipulated to scrub. Once the patient is sterilized, the excess antiseptic solution is manipulated into the drainage pouch.

In one embodiment, the antiseptic solution is introduced into the application bag through an open end of the application bag. In another embodiment, an antiseptic pouch within the application bag may be ruptured to disinfect the patient.

A medical grade deformable seal is affixed to the open end(s) of the application bag. The seal fits snuggly around the patient's body forming a seal between the interior of the bag and patient's body. The seals prevents the release of the antiseptic until drained into the drainage pouch and the drainage pouch is removed.

### BRIEF DESCRIPTION OF THE DRAWINGS

In the detailed description of the preferred embodiments presented below, reference is made to the accompanying drawings.

FIG. 1A is a plan view of an embodiment of the invention showing a removable drainage pouch.

FIG. 1B is an exploded perspective view of an embodiment of the invention showing a removable drainage pouch.

FIG. 1C is a detailed view of a preferred embodiment of a textured surface of the invention.

FIG. 1D is a detailed view of an alternate embodiment of a textured surface of the invention.

FIG. 1E is a plan view of an embodiment of the invention with a shipping insert.

FIG. 2A is a plan view of an alternate embodiment of the invention showing a resealable closure.

FIG. 2B is a perspective view of an alternate embodiment of the invention showing a resealable closure.

FIG. 3A is a plan view of a preferred embodiment of the invention showing use with multiple appendages.

FIG. 3B is an exploded perspective view of a preferred embodiment of the invention showing detachable compartments.

FIG. 4A is a plan view of a preferred embodiment of the invention.

FIG. 4B is an exploded perspective view of an embodiment of the invention showing a detachable compartment.

FIG. 5 is a flowchart of the steps involved in use of certain preferred embodiments.

### DETAILED DESCRIPTION OF THE INVENTION

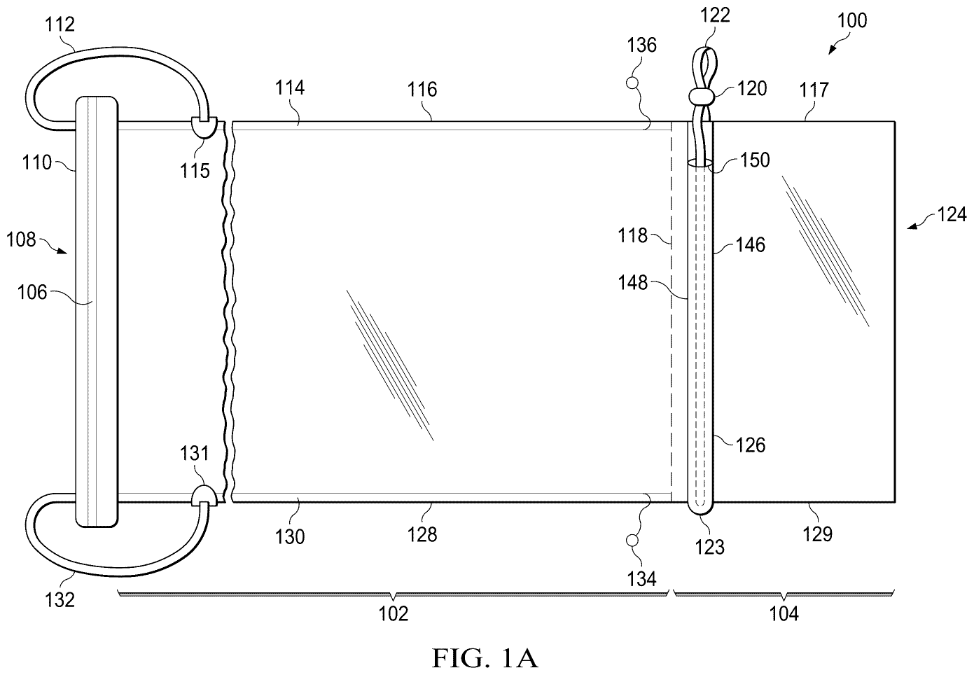

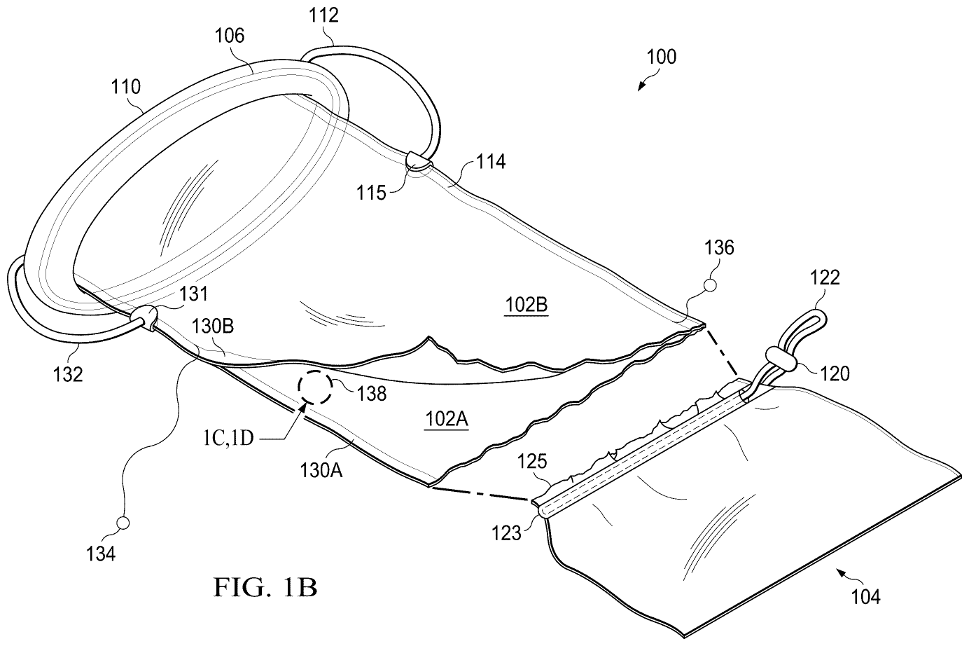

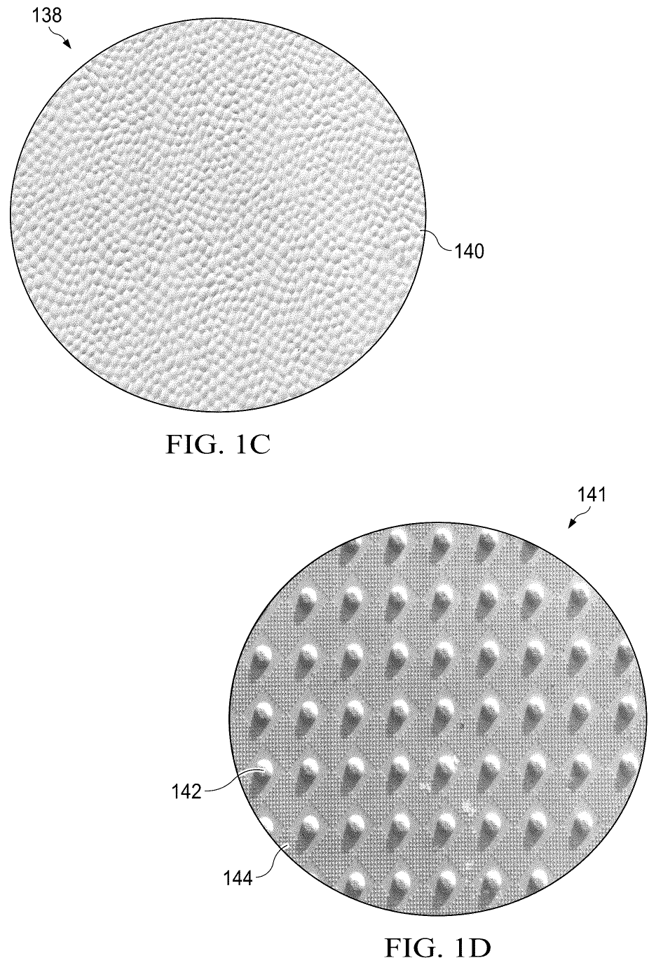

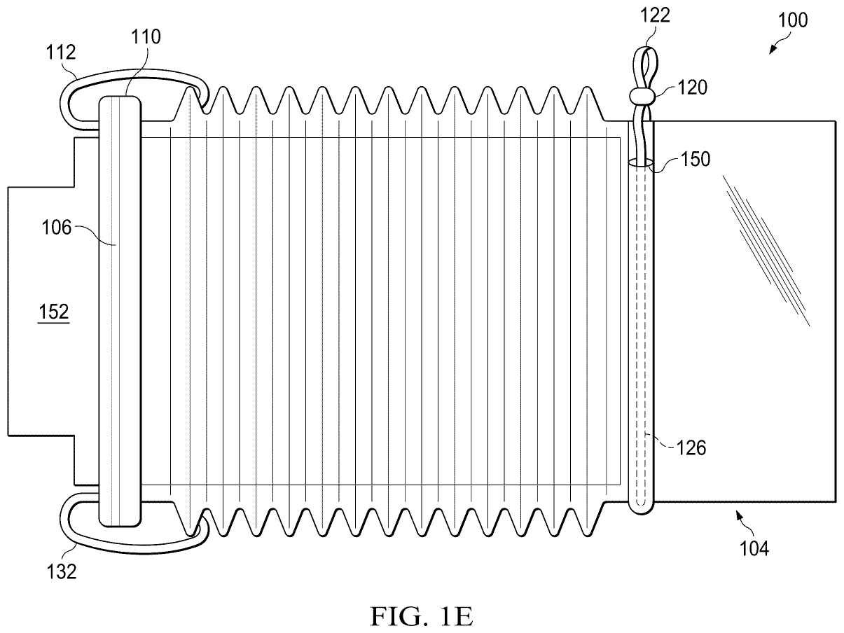

Referring then to FIGS. 1A and 1B a preferred embodiment of application bag 100 is shown. In all following embodiments all tolerances and ranges are ±20%, unless otherwise specified.

Application bag 100 is comprised of a flexible plastic generally tubular in shape having closed sides 116 and 128, closed distal end 124, and open end 108. In a preferred embodiment, application bag 100 is made of 40-60 mil HDPE (high density polyethylene) which is typically translucent. In other embodiments, the application bag can be made out of LDPE (low density polyethylene) and is less opaque but not completely transparent. In other embodiments, LLDP (linear low density polyethylene) can be employed where a higher structural rigidity is required by the cleaning process. Other flexible sheeting can be used so long as it is sealable and inert with respect to the antiseptic solution.

In a preferred embodiment, application bag 100 is formed from two identical or nearly-identical sized rectangular sheets. Distal end 124 is fused closed by inductive welding. Open end 108 includes flexible seal 110.

Seal 110 is preferably comprised of a medical grade closed cell, low density PVC foam circumferentially surrounded by elastic band 106. Optionally, seal 110 includes a suitable adhesive for direct application to the exterior of an appendage or extremity. Elastic band 106 is positioned around the exterior of flexible seal 110 and releasably secures the foam around an appendage to form an airtight seal. Different parts of the body can be decontaminated using application bag. The dimensions of the application bag and elastic band vary depending on the part of the body being decontaminated. Application bag 100 should fit loosely around the body part placed inside application bag 100. Typical dimensions are about 100 centimeters in length and about 60 centimeters width forming a bag with a working diameter of about 20 centimeters. The length of drainage pouch 104 is typically about 10 cm, but these dimensions can vary.

Handle 112 and handle 132 are inductively welded to the application bag at open end 108 and along sides 116 and 128 adjacent flexible seal 110. Handles 112 and 132 are preferably comprised of a flexible plastic strap material having a high structural rigidity, such as 60-80 mil LLDP. In a preferred embodiment, handles 132 and 112 include support buttresses 131 and 115, respectively. The buttresses are heat welded to the application bag adjacent the foam seal. In a preferred embodiment, the buttresses are 50 mil HDPE sheet material.

Side 116 is sealed or fused by latitudinal seal 114 and heat weld 117. Side 128 is sealed or fused by latitudinal seal 130 and heat weld 129. In a preferred embodiment, latitudinal seals 114 and 130 are semi-permanent airtight seals that may be broken to separate first portion 102A from second portion 102B of application portion 102.

In a preferred embodiment, latitudinal seals 114 and 130 are heat welds having imbedded pull strings 136 and 134, respectively. Pull string 134 is designed to tear seal 130 by separating first seal portion 130A from second seal portion 130B, as shown in FIG. 1B. Likewise, pull string 136 is designed to tear seal 114.

In another embodiment, latitudinal seals 114 and 130 are comprised of suitably aggressive yet releasable medical grade adhesive which allows for a resealable closure between the two. In yet another embodiment, the first portion and second portion of seals 114 and 130 are comprised of a Ziploc® type resealable zipper closure.

Application bag is further comprised of cut line 118. Preferably, cut line 118 is a visible line on or embedded in the surface of the plastic sheeting which demarcates the separation line between application portion 102 and drainage pouch 104. Preferably, cut line 118 is located approximately 7-10 cm from distal end 124. Cut line 118 runs generally parallel to open end 108 and distal end 124. In a preferred embodiment, cut line 118 is located at and perpendicular to the transition between seal 114 and weld 117, and the transition between seal 130 and weld 129. In a preferred embodiment, cut line 118 is a dotted line running around the circumference of application bag 100. However, other demarcations may be used.

Drainage pouch 104 is adjacent cut line 118 and distal end 124. Drainage pouch 104 includes double layer enclosure 126 which runs generally parallel to cut line 118 around most of the circumference of the application bag. Double layer enclosure 126 includes heat welded seal 146, heat welded seal 148, which form hollow tube 123. Hollow tube 123 is terminated by two openings, such as opening 150. Drawstring 122 is movably positioned in hollow tube 123 and extends out of the openings. Drawstring 122 includes clamp 120 which is utilized to cinch the drainage pouch closed.

In use, antiseptic solution used to disinfect an appendage is drained into the drainage pouch and contained there by pulling the drawstring and cinching off the clamp to close opening 125. The drainage pouch may then be removed from the application bag, as will be further described.

The interior of application portion 102 includes textured surface 138 to increase the effectiveness of the application of the antiseptic to the patient, as will be further described.

Referring then to FIG. 1C, textured surface 138 is shown. In a preferred embodiment, textured surface 138 is comprised of a plurality of irregular dimples 140 which extend from the interior or outside surface. In a preferred embodiment, the dimples are irregularly spaced and shaped. In other embodiments, the dimples may be only irregularly shaped or irregularly sized. The rough textured surface is used to scrub the surgical site and facilitate the removal of bacterial colonies. Irregular dimples 140 provide a friction coefficient sufficient to adequately sterilize a wound or surface area but gentle enough to prevent causing unnecessary irritation to wounds or sensitive areas.

Referring then to FIG. 1D, alternative textured surface 141 is shown. Alternate textured surface 141 is comprised of a plurality of large dimples 142 and small dimples 144. In one embodiment, the large and small dimples are regularly spaced and sized. In other embodiments, the either the large or small dimples may be irregular in spacing or size. The use of dimples of various sizes results in a higher friction coefficient which may be utilized to scrub the surgical site and facilitate the removal of bacterial colonies when more aggressive sterilization is required, such as when the surgical site is more soiled or prone to severe bacterial infections.

Referring then to FIG. 1E, a preferred configuration for shipment and storage of application bag 100 is shown. In a preferred embodiment, the application bag is stored in a compressed accordion format to increase the ease of deployment. In a preferred embodiment, in the compressed format flexible seal 110 is positioned around removable rigid insert 152. In another embodiment, the application bag may also be stored in a flat format to increase the ease of storage.

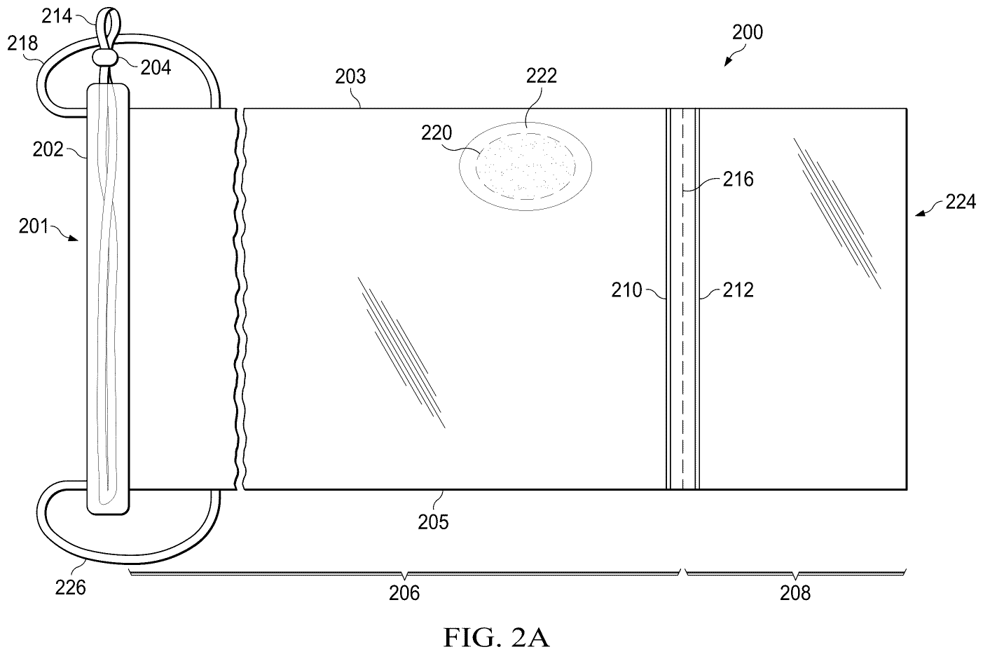

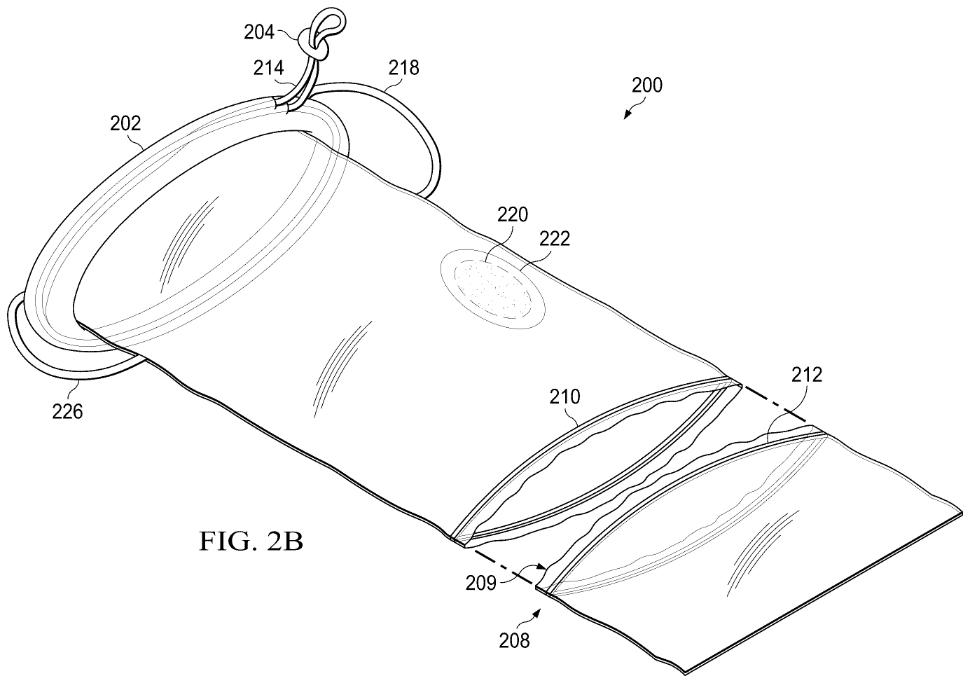

Referring then to FIGS. 2A and 2B an alternate embodiment of application bag 200 is shown.

Application bag 200 is a generally tubular in shape having closed sides 203 and 205, closed distal end 224, and open end 201. In a preferred embodiment, application bag 200 is formed of a flexible plastic sheet having a textured interior surface, as previously described. Distal end 224, side 203, and side 205 are typically fused closed by inductive welding. Open end 201 includes flexible seal 202.

Seal 202 is preferably comprised of a medical grade closed cell, low density PVC foam, as previously described. In a preferred embodiment, seal 202 includes drawstring 214. Drawstring 214 includes spring loaded friction clamp 204 which is utilized to secure seal 202 around an appendage and which forms an airtight seal. In one embodiment drawstring 214 is integrally formed with the seal. In another embodiment, the drawstring is secured to the seal separate elongated tube heat welded to the application bag exterior surface. The dimensions of the application bag vary depending on the part of the body being decontaminated, as previously described.

Handle 218 and handle 226 are inductively welded to the application bag at open end 201 and along sides 203 and 205 adjacent flexible seal 202. Handles 218 and 226 are comprised of a flexible plastic having a high structural rigidity, such as LLDP.

Application bag 200 includes cut line 216. Line 216 marks the connection line between application portion 206 and drainage pouch 208. In a preferred embodiment, line 216 is located approximately 7-10 cm from distal end 224. Line 216 runs generally parallel to open end 201 and distal end 224. In a preferred embodiment, line 216 is a dotted line running around the circumference of application bag 200. However, other demarcations may be used.

Application bag 200 is further comprised of seal 210 and seal 212. Seals 210 and 212 are airtight resealable Ziploc® type closures located adjacent and parallel to line 216. Seal 210 is located approximately 0.5 cm proximal to cut line 216. Seal 212 is located approximately 0.5 cm distal to line 216.

Drainage pouch 208 is adjacent seal 212 and distal end 224. In use, after disinfecting an appendage, antiseptic solution is drained into the drainage pouch through opening 209. Seals 210 and 212 are secured thereby sealing opening 209 closed and the drainage pouch is removed by cutting along line 216, as will be further described.

Optionally, application bag 200 is further comprised of antiseptic pouch 220. Antiseptic pouch 220 is comprised of a thin layer of plastic hermetically sealed to the interior surface of application bag 200 via seal 222. The pouch may be ruptured to release a premeasured amount of antiseptic solution into the application bag after securing the bag around an appendage.

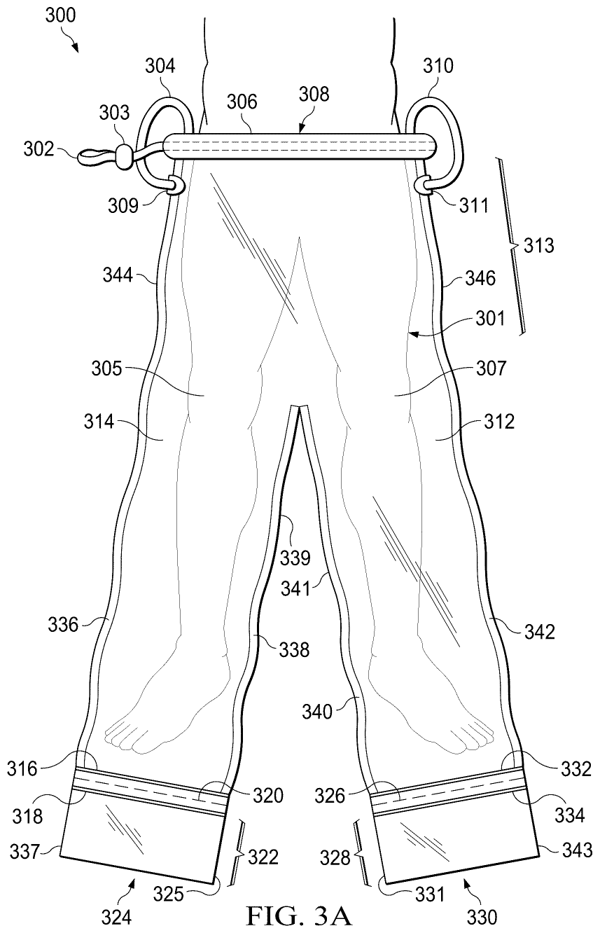

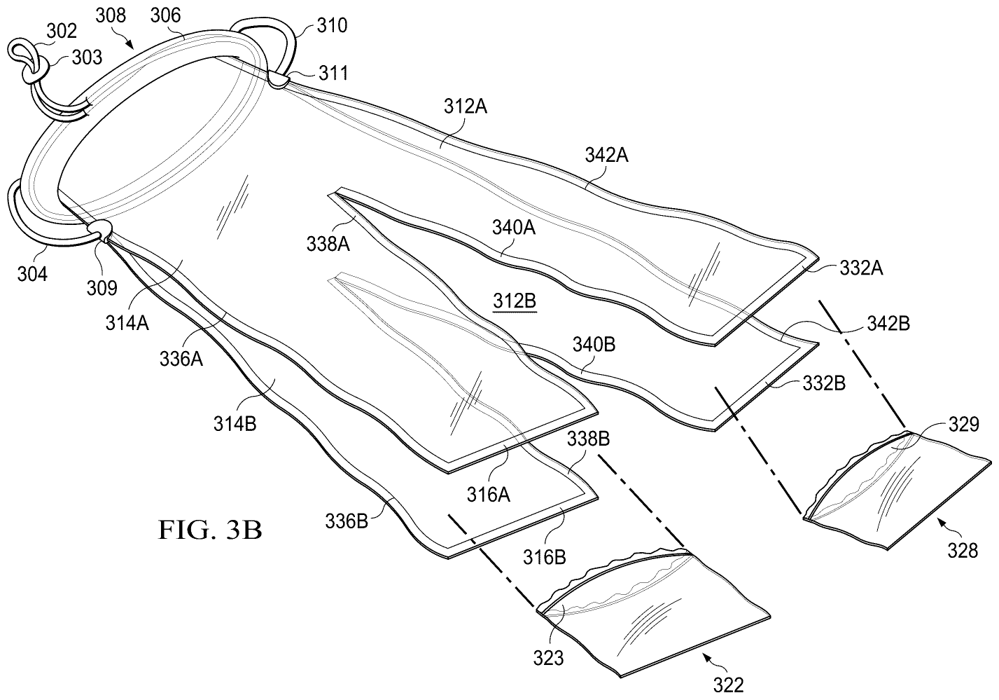

Referring then to FIGS. 3A and 3B a preferred embodiment of application bag 300 is shown.

Application bag 300 is comprised of a generally tubular “Y” shape having leg portion 314, leg portion 312, trunk portion 313, and open end 201, as will be further described. In a preferred embodiment, application bag 300 is made of flexible translucent or transparent plastic sheeting having a textured interior surface, as previously described.

Open end 308 includes flexible seal 306. Seal 306 is preferably comprised of a medical grade closed cell, low density PVC foam, as previously described. In a preferred embodiment, seal 306 includes drawstring 302. Drawstring 302 includes clamp 303 which is utilized to secure seal 306 around patient 301 to form an airtight seal. In one embodiment, drawstring 302 is integrally formed with the seal. In another embodiment, the drawstring is secured to the seal using a double sided enclosure heat welded to the application bag. The dimensions of the application bag vary depending on the patient size. Typical embodiments include a trunk portion of about 60 cm in length, and 60 cm in width, and leg portions of about 180 cm in length and about 50 cm width.

Handle 304 and handle 310 are inductively welded to the application bag at open end 308 and along exterior sides 344 and 346 adjacent seal 306. Handles 304 and 310 are comprised of a flexible plastic having a high structural rigidity, such as LLDP. In a preferred embodiment, handles 304 and 310 include support buttresses 309 and 311, respectively. The buttresses are heat welded to the application bag adjacent the foam seal.

In a preferred embodiment, application bag 300 is formed from two identical or nearly-identical sized and shaped sheets. Leg portion 314 is formed from exterior side 344, distal end 324, and interior side 339. Leg portion 314 is formed from exterior side 344, distal end 324, and interior side 339. Distal ends 324 and 330 are preferably fused by inductive welding or a suitable medical grade adhesive.

Exterior side 344 is sealed or fused by exterior seal 336 and heat weld 337. Interior side 339 is sealed or fused by interior seal 338 and heat weld 325. Exterior side 346 is sealed or fused by exterior seal 342 and heat weld 343. Interior side 341 is sealed or fused by interior seal 340 and heat weld 331.

In a preferred embodiment, seals 336 and 338 are semi-permanent airtight seals that may be broken to separate first portion 314A from second portion 314B of leg portion 314. Likewise, seals 340 and 342 are semi-permanent airtight seals that may be broken to separate third portion 312A from fourth portion 312B of leg portion 312. In one embodiment, seals 336, 338, 340, and 342 are heat welds having imbedded pull strings, as previously described. In another embodiment, seals 336, 338, 340, and 342 are comprised of suitably aggressive yet releasable adhesive which allows for a resealable closure. In yet another embodiment, first portions 336A, 338A, 340A, and 342A, and second portions 336B, 338B, 340B, and 342B of seals 336, 338, 340, and 342 are comprised resealable zipper closures.

Application bag 300 includes cut lines 320 and 326. Line 320 marks the juncture point of leg portion 314 and drainage pouch 322. In a preferred embodiment, line 320 is located approximately 7-10 cm from distal end 324. Line 320 is generally parallel to distal end 324. Line 326 marks the juncture point of leg portion 312 and drainage pouch 328. In a preferred embodiment, line 326 is located approximately 7-10 cm from distal end 330. Line 326 is generally parallel to distal end 330. In a preferred embodiment, lines 320 and 326 are dotted lines running around the circumference of leg portions 314, and 312, respectively. However, other demarcations may be used.

Application bag 300 is further comprised of seal 316 and seal 318. Seals 316 and 318 are airtight resealable Ziploc® type closures located adjacent and parallel to line 320. Seal 316 is located approximately 0.5 cm proximal to line 320. Seal 318 is located approximately 0.5 cm distal to line 320. Drainage pouch 322 is adjacent seal 318 and distal end 324. In use, after disinfecting an appendage, antiseptic solution is drained into drainage pouch 322 through opening 323. Seals 316 and 318 are secured thereby sealing opening 323, and the drainage pouch is removed by cutting along line 320, as will be further described.

Application bag 300 is further comprised of seal 332 and seal 334. Seals 332 and 334 are airtight resealable Ziploc® type closures located adjacent and parallel to line 326. Seal 332 is located approximately 0.5 cm proximal to line 326. Seal 334 is located approximately 0.5 cm distal to line 326. Drainage pouch 328 is adjacent seal 334 and distal end 330. In use, after disinfecting an appendage, antiseptic solution is drained into drainage pouch 328 through opening 329. Seals 332 and 334 are secured thereby sealing opening 329, and the drainage pouch is removed by cutting along line 326, as will be further described. Thereafter, appendage 305 may be exposed in an operating theater by separating seals 338, 316, and 336 into first portions 338A, 316A, and 336A from second portions 338B, 316B, and 336B, respectively. Likewise, appendage 307 may be exposed in an operating theater by separating seals 342, 332, and 340 into first portions 342A, 332A, and 340A from second portions 342B, 332B, and 340B, respectively.

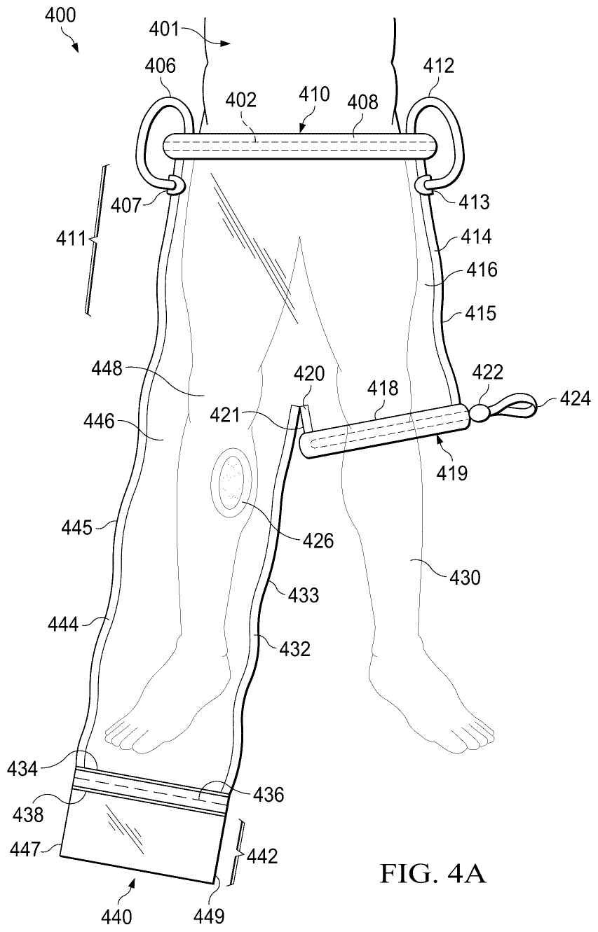

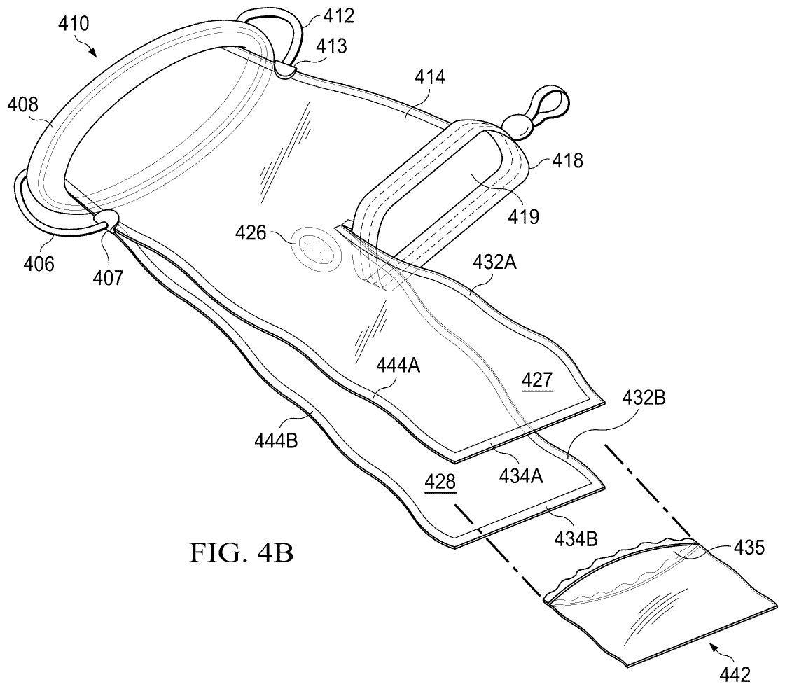

Referring then to FIGS. 4A and 4B an embodiment of application bag 400 is shown.

Application bag 400 is comprised of a generally tubular asymmetrical “Y” shape having leg portion 446, cropped portion 416, trunk portion 411, and open end 410, as will be further described. In a preferred embodiment, application bag 400 is made of flexible plastic sheeting having a textured interior surface, as previously described.

Open end 410 includes seal 408. Seal 408 is preferably comprised of a medical grade closed cell, low density PVC foam and elastic band 402, as previously described. Optionally, seal 408 includes a suitable adhesive for direct application to the patient. Elastic band 402 is positioned around the exterior of seal 408 and compresses the foam securely around patient 401 to form an airtight seal. The dimensions of the application bag and elastic band vary depending on the patient size. Application bag 400 ideally is secured around the appendages inside application bag 400.

Handle 406 and handle 412 are inductively welded to the application bag at open end 410 and along exterior sides 445 and 415 adjacent seal 408. Handles 406 and 412 are comprised of a flexible plastic strap material having a high structural rigidity, such as LLDP. In a preferred embodiment, handles 406 and 412 include support buttresses 407 and 413, respectively. The buttresses are heat welded to the application bag adjacent the foam seal, as previously described.

In a preferred embodiment, application bag 400 is formed from two identical or nearly-identical sized and shaped flat plastic sheets. Leg portion 446 is formed from exterior side 445, distal end 440, and interior side 433. Distal end 440 is fused closed by inductive welding. Cropped portion 416 is formed from exterior side 415, opening 419, and interior side 421.

Exterior side 445 is sealed or fused by exterior seal 444 and heat weld 447. Interior side 433 is sealed or fused by interior seal 432 and heat weld 449. Exterior side 415 is sealed or fused by exterior seal 414. Interior side 421 is sealed or fused by interior seal 420.

In a preferred embodiment, seals 444 and 432 are semi-permanent airtight seals that may be broken to separate first portion 427 from second portion 428 of leg portion 446. In one embodiment, seals 444 and 432 are heat welds having imbedded pull strings, as previously described. In another embodiment, seals 444 and 432 are comprised of suitably aggressive yet releasable medical grade adhesive which allows for a resealable closure. In yet another embodiment, first portions 444A and 432A, and second portions 444B and 432B of seals 444 and 432 are comprised of resealable Ziploc® type closures.

In a preferred embodiment, seals 420 and 414 are heat welds. Alternate airtight seals may be used, as previously described.

Application bag 400 includes cut line 436. Line 436 marks the juncture point of leg portion 446 and drainage pouch 442. In a preferred embodiment, line 436 is located approximately 7-10 cm from distal end 440. Line 436 is generally parallel to distal end 440. In a preferred embodiment, line 436 is a dotted line extending around the circumference of leg portion 446. However, other demarcations may be used.

Application bag 400 is further comprised of seal 434 and seal 438. Seals 434 and 438 are airtight resealable Ziploc® type closures located adjacent and parallel to line 436. Seal 434 is located approximately 0.5 cm proximal to line 436. Seal 438 is located approximately 0.5 cm distal to line 436. Drainage pouch 442 is adjacent seal 438 and distal end 440. In use, after disinfecting appendage 448, antiseptic solution is drained into the drainage pouch through opening 435. Seals 434 and 438 are secured thereby closing opening 435, and the drainage pouch is removed by cutting along line 436, as will be further described. Thereafter, appendage 448 may be exposed in an operating theater by separating seals 432, 434, and 444 into first portions 432A, 434A, and 444A from second portions 432B, 434B, and 444B, respectively.

Cropped portion is further comprised of opening 419. Opening 419 includes seal 418. Seal 418 is preferably comprised of a medical grade closed cell, low density PVC foam, as previously described. In a preferred embodiment, seal 418 includes drawstring 424. Drawstring 424 includes clamp 422 which is utilized to secure seal 418 around appendage 430 to form an airtight seal. In one embodiment, drawstring 424 is integrally formed with the seal. In another embodiment, the drawstring is secured to the seal using a double sided enclosure heat welded to the application bag. The dimensions of the application bag vary depending on the patient size, as previously described.

Optionally, application bag 400 is further comprised of antiseptic pouch 426. Antiseptic pouch 426 is comprised of a thin layer of plastic hermetically sealed to the interior surface of application bag 400, as previously described.

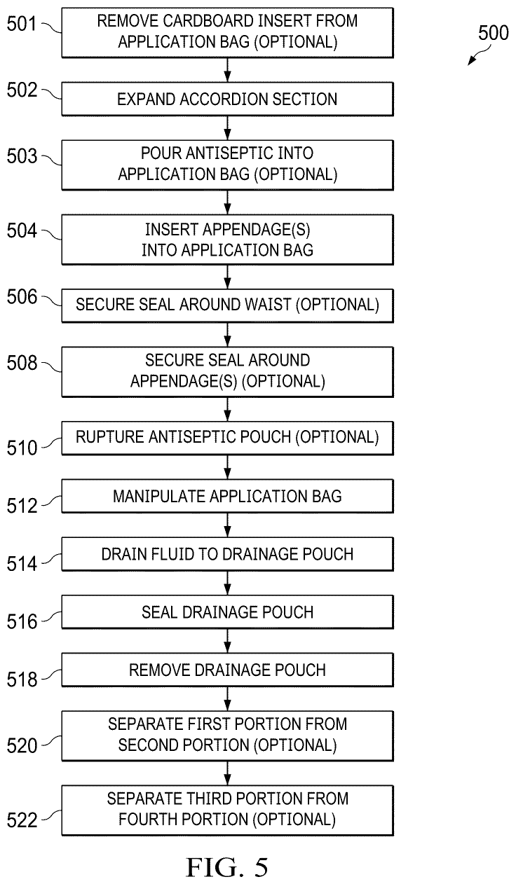

Referring now to FIG. 5, the steps for preferred method 500 of use of the various embodiments of the application bag are shown.

At step 501, insert 152 is removed from the application bag.

At step 502, the accordion section is expanded. In embodiments where the application bag is not shipped and stored with an insert, steps 501 and 502 are omitted.

At step 503, antiseptic fluid is introduced to the interior of the application bag through an open end.

At step 504, the subject appendage or appendages, as applicable, are inserted into the application bag through an open end. Steps 502 and 504 may be reversed in sequence. In other words, the appendage(s) may be inserted through the open end prior to the antiseptic solution. Likewise, steps 503 and 504 may be reversed.

At step 506, optionally, the application bag is sealed around a patient waist.

At step 508, the application bag is sealed around the appendage(s).

At step 510, when applicable, the thin membrane of the antiseptic pouch is ruptured to release the solution into the application bag.

At step 512, the application bag is manipulated to apply the antiseptic fluid to the extremity and use the textured interior surface to dislodge bacterial colonies.

At step 514, the excess antiseptic fluid is drained into the drainage pouch after disinfecting the extremity with the antiseptic fluid. This is generally achieved by manipulating the application bag.

At step 516, the drainage pouch is sealed. The drainage pouch may be sealed by pulling a drawstring or by connecting the resealable zippers.

At step 518, the drainage pouch is removed by cutting the application bag along the cut line.

At step 520, optionally, an appendage may be exposed by separating a first portion from a second portion without disturbing the appendage. At step 522, optionally, a second appendage may be exposed by separating a third portion from a fourth portion without disturbing the appendage. In a preferred embodiment, the first portion is separated from the second portion using a pull string which severs a seal between the two portions. Likewise, the third portion may be separated from the fourth portion using a pull string. In an alternate embodiment, the seals are resealable and may be separated by applying opposing forces on the connected portions.

With the appendage disinfected and exposed, the seal may remain in place while physicians move forward with surgery on the appendage. Post-surgery, the application bag may be reassembled to protect the surgical site from bacteria before transferring the patient from the surgical environment to the recovery area. One of skill in the art will recognize that these steps may also be applied to other embodiments of the application bag as required. The embodiments have been described in detail with particular reference to certain preferred embodiments thereof, but it will be understood that variations and modifications can be affected within the scope of the embodiments, especially to those skilled in the art.

1. A device for applying an antiseptic solution to a patient comprising:
a flexible tubular container, configured to surround an appendage of the patient, having an application portion and a drainage portion;
a first opening, having a pliable seal, ductedly connected to the application portion; and
a first closure means, detachably connecting the application portion and the drainage portion, for isolating and removing the antiseptic solution.

2. The device of claim 1, further comprising:
a tightening means, integrated into the pliable seal, for sealing the flexible tubular container to the appendage.

3. The device of claim 1, further comprising:
a second closure means, adjacent the first closure means, for sealing the application portion.

4. The device of claim 3, wherein the second closure means further comprises a resealable zipper.

5. The device of claim 3, wherein the second closure means further comprises:
a drawstring; and
a clamp secured to the drawstring.

6. The device of claim 1, wherein the first closure means further comprises a resealable zipper.

7. The device of claim 1, wherein the first closure means further comprises:
a drawstring; and
a clamp secured to the drawstring.

8. The device of claim 1, wherein the flexible tubular container further comprises:
a first longitudinal closure, adjacent the application portion; and
a second longitudinal closure, adjacent the application portion, and diametrically opposed to the first longitudinal closure.

9. The device of claim 8, further comprising:
a first pull string means, incorporated into the first longitudinal closure, for opening the first longitudinal closure.

10. The device of claim 9, further comprising:
a second pull string means, incorporated into the second longitudinal closure, for opening the second longitudinal closure.

11. The device of claim 1, further comprising:
a first handle, connected to the pliable seal and the application portion; and
a second handle, connected to the pliable seal and the application portion, diametrically opposed to the first handle.

12. The device of claim 11, further comprising:
a first buttress pad, connecting the first handle and the application portion; and
a second buttress pad, connecting the second handle and the application portion.

13. The device of claim 1, wherein the flexible tubular container is formed of an HDPE sheet material.

14. The device of claim 13, wherein the HDPE sheet material includes an inner textured surface.

15. The device of claim 1, wherein the application portion further comprises:
an interior surface; and
a flexible rupturable container, filled with an antiseptic solution, fixed to the interior surface.

16. The device of claim 1, further comprising:
a planar packaging insert, removably positioned in the flexible tubular container, through the first opening and inside the first closure means.

17. A device for applying an antiseptic solution to a patient comprising:
a flexible y-shaped container, having an application portion, configured to surround a lower appendage of the patient;
the application portion, having a trunk section, ductedly connected to a first leg section and a second leg section;
a first opening, having a first pliable seal, ductedly terminating the trunk section;
a first drainage portion, removably connected to the first leg section; and
a first separatable closure means, between the trunk section and the first drainage portion, for sealing the first drainage portion and detachably removing the antiseptic solution.

18. The device of claim 17, further comprising:
a first tightening means, connected to the first pliable seal, for securing the trunk section to the patient.

19. The device of claim 17, further comprising:
a second separatable closure means, between the trunk section and the first drainage portion, for sealing the first leg section.

20. The device of claim 17, further comprising:
a second opening, having a second pliable seal, ductedly terminating the second leg section.

21. The device of claim 19, further comprising:
a second drainage portion, removably connected to the second leg section; and
a third separatable closure means, between the trunk section and the second drainage portion, for sealing the second drainage portion and detachably removing the antiseptic solution.

22. The device of claim 21, further comprising:
a fourth separatable closure means, between the trunk section and the second drainage portion, for sealing the second leg section.

23. The device of claim 22, wherein the first separatable closure means further comprises a first resealable zipper and the second separatable closure means further comprises a second resealable zipper.

24. The device of claim 17, wherein the first leg section further comprises a first longitudinal closure, sealing the application portion and a second longitudinal closure, sealing the application portion and diametrically opposed to a first longitudinal closure, and the second leg section further comprises a third longitudinal closure, sealing the application portion and a fourth longitudinal closure, sealing the application portion and diametrically opposed to the third longitudinal closure.

25. The device of claim 17, further comprising:
a set of handles, connected to the first pliable seal, for positioning the flexible y-shaped container on the patient.

26. The device of claim 17, wherein the flexible y-shaped container is formed of an HDPE material.

27. A method of applying an antiseptic solution to a patient comprising:
inserting an appendage of the patient into a flexible tubular container;
introducing the antiseptic solution into the flexible tubular container;
sealing the flexible tubular container around the appendage;
cleaning the appendage with the antiseptic solution;
draining the antiseptic solution into a detachable drainage pouch of the flexible tubular container;
sealing the detachable drainage pouch; and
physically separating the detachable drainage pouch from the flexible tubular container.

28. The method of claim 27, further comprising:
resealing the flexible tubular container.

29. The method of claim 27, further comprising:
rupturing a first longitudinal seal in the flexible tubular container; and
rupturing a second longitudinal seal in the flexible tubular container;
wherein the appendage is exposed.

30. The method of claim 27, further comprising:
removing a planar packing insert from the flexible tubular container; and
expanding the flexible tubular container, from a corrugated state to an expanded state, before inserting the appendage of the patient.

31. A method of applying an antiseptic solution to a patient, the patient having a trunk and lower extremities comprising:
inserting the lower extremities of the patient into a y-shaped flexible container;
sealing the y-shaped flexible container about the trunk of the patient;
introducing the antiseptic solution into the y-shaped flexible container;
cleaning a first leg of the patient with the antiseptic solution;
draining a first portion of the antiseptic solution into a first detachable drainage pouch of the y-shaped flexible container;
sealing the first detachable drainage pouch; and
physically separating the first detachable drainage pouch from the y-shaped flexible container.

32. The method of claim 31, further comprising:
resealing the y-shaped flexible container.

33. The method of claim 31, further comprising:
sealing the y-shaped flexible container about a second leg of the patient before introducing the antiseptic solution.

34. The method of claim 31, further comprising:
cleaning a second leg of the patient with the antiseptic solution;
draining a second portion of the antiseptic solution into a second detachable drainage pouch of the y-shaped flexible container;
sealing the second detachable drainage pouch; and
physically separating the second detachable drainage pouch from the y-shaped flexible container.
